(19) United States
(12) Patent Application Publication
(10) Pub. No.: US 2014/0296479 A1
(43) Pub. Date: Oct. 2, 2014

(54) D-APTIDE AND RETRO-INVERSO APTIDE WITH MAINTAINED TARGET AFFINITY AND IMPROVED STABILITY

(71) Applicants: Sang-Yong Jon, Gwangju (KR); Sung-Hyun Kim, Gwangju (KR)

(72) Inventors: Sang-Yong Jon, Gwangju (KR); Sung-Hyun Kim, Gwangju (KR)

(73) Assignee: GWANGJU INSTITUTE OF SCIENCE AND TECHNOLOGY, Gwangju (KR)

(21) Appl. No.: 14/233,724

(22) PCT Filed: Apr. 6, 2012
(86) PCT No.: PCT/KR2012/002631

§ 371 Date: Jun. 16, 2014

(30) Foreign Application Priority Data
Apr. 8, 2011 (KR) 10-2011-0032928

(51) Int. Cl. C07K 1/113; C07K 14/00

(52) U.S. Cl. CPC C07K 1/1136; C07K 14/00; C07K 14/001
USPC 530/324; 530/333; 506/2

(57) ABSTRACT

The present invention is characterized by a D-Aptamer-Like Peptide (D-Aptide) or retro-inverso Aptide which specifically binds to a target comprising: (a) a structure stabilizing region comprising parallel, antiparallel or parallel and antiparallel D-amino acid strands with interstrand noncovalent bonds; and (b) a target binding region I and a target binding region II comprising randomly selected n and m D-amino acids, respectively, and coupled to both ends of the structure stabilizing region. The D-Aptide or retro-inverso Aptide has the sequence of the same or opposite direction to L-Aptide, wherein the stability to proteases is improved while maintaining the affinity to a target compared with L-Aptide. The D-Aptide of the present invention has substantially the same target affinity and a remarkably improved stability compared with L-Aptide which is different from a general technical knowledge.

### CROSS REFERENCE TO RELATED APPLICATIONS AND CLAIM OF PRIORITY

This patent application is a National Phase application under 35 U.S.C. §371 of International Application No. PCT/KR2012/002631, filed 6 Apr. 2012, which claims priority to Korean Patent Application number 10-2011-0032928, filed 8 Apr. 2011, entire contents of which are incorporated herein by reference.

### BACKGROUND

1. Field of the Invention

The present invention is related to an Aptamer-Like Peptide (Aptide) consisting of D-amino acids whose target affinity is maintained and stability is improved, i.e. a D-Aptide and a retro-inverso Aptide.

2. Description of the Related Art

Using high-specification and high-affinity of antigen-antibody reaction and applying a variety of antibodies capable of discriminating 10 million antigens, numerous antibody products including diagnostics and therapeutics have been developed nowadays. Twenty one monoclonal antibodies have been approved by FDA until now, and antibodies such as Rituximab and Herceptin have been proved to have an excellent efficacy over 50% of subjects who exhibit no response to other therapies. In practice, the utilization of monoclonal antibodies results in successful clinic treatment for cancers including lymphoma, colorectal cancer or breast cancer. Whole market size of therapeutic antibodies might be evaluated to be in an annual average of 20% growth rate from 10 billion dollars in 2004 to 30 billion dollars in 2010 and predicted to be increased in a geometrical progression.

There has been emerging focus on development of new drug using antibody because of short development period of drug, economical investment cost and feasible prediction of adverse effects. Additionally, antibody as a herb medicine has no influence on a human body and is beneficial to a subject since it has half-life much longer than drugs with a low molecular weight.

In spite of these availabilities, monoclonal antibodies may induce severe allergic or hypersensitive responses in human body due to recognition as a foreign antigen. Furthermore, clinical utilization of a monoclonal antibody with an anti-cancer activity has the following drawbacks: high therapeutics cost due to high production cost, and expensive licensing fees because intellectual property rights protect widespread techniques such as culture and purification method of antibodies.

Accordingly, to overcome these problems, it is earlier beginning to develop antibody alternatives. The antibody alternatives are designed as a recombinant protein having constant and variable domain like an antibody, of which the size is small and a particular region of a stable protein is replaced by random amino acid sequence, leading to produce a library, and the library is utilized for screening a target molecules to isolate a molecule with high affinity and excellent specificity.

For example, it has been reported that avimer and affibody of antibody alternatives have a superior affinity to a target molecule in picomole level. Generally, the small-sized and stable antibody alternatives have been reported to penetrate into cancer cells in a feasible manner and to induce immune responses in a low level. First of all, the antibody alternatives may avoid antibody patent barriers and have excellent advantages such as low production cost and feasible massive purification from bacteria.

Currently, 40 antibody alternatives have been known, and the example of antibody alternatives commercially attempted in ventures or international pharmaceuticals includes fibronectin type III domain, lipocalin, LDLR-A domain, crystalline, protein A, ankyrin repeat or BPTI protein, which have high affinity to a target molecule in the level of picomole. Of them, FDA clinic experiments for adnectin, avimer or Kunitz domain are on-going at present.

Meanwhile, the present inventors have already suggested an Aptamer-Like Peptide (bipodal peptide binder: BPB) as an antibody alternative (WO 2010/047515). The term ‘BPB’ has been changed into “Aptide (Aptamer-Like Peptide)”.

The Aptide is an antibody alternative, and has very low molecular weight and very high affinity to a target as a result of its unique structure.

However, it is still required to improve in vivo stability of the Aptide to apply it clinically. Such improvement in in vivo stability should be achieved without damage to the affinity to a target.

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY

The present inventors have made intensive studies to develop technique for improving stability of Aptamer-Like Peptides (Aptides) with its target affinity maintained. As a result, we have discovered that where Aptides were prepared using D-amino acids which were known in the art that they decrease the affinity to a target, prepared D-Aptides have enhanced stability with binding affinity almost identical to L-Aptides (Aptide consisting of L-amino acids).

Therefore, it is an aspect of this invention to provide a method for enhancing stability of an Aptide consisting of L-amino acids (L-Aptide) to a protease while maintaining the affinity to a target.

It is another aspect of this invention to provide a D-Aptamer-Like Peptide (D-Aptide) having enhanced stability to a protease with its affinity to a target maintained in comparison with L-Aptide.

Other aspects and advantages of the present invention will become apparent from the detailed description to follow taken in conjugation with the appended claims and drawings.

In one aspect of this invention, there is provided a method for enhancing stability of an Aptide consisting of L-amino acids (L-Aptide) to a protease while maintaining the affinity to a target, comprising preparing an Aptamer-Like Peptide (Aptide) comprising: (i) a structure stabilizing region comprising parallel, antiparallel, or parallel and antiparallel amino acid strands with interstrand non-covalent bonds; and (ii) a target binding region I bound to one terminus of both termini of the structure stabilizing region and a target binding region II bound to the other terminus of the both termini of the structure stabilizing region, wherein the target binding region I and the target binding region II comprises randomly selected n and m amino acids, respectively, and wherein the structure stabilizing region, the target binding region I and the target binding region II are prepared with D-amino acids, thereby preparing a D-Aptide or a retro-inverso Aptide.

In another aspect of this invention, there is provided a D-Aptamer-Like Peptide (D-Aptide) which specifically binds to a target, comprising: (a) a structure stabilizing region comprising parallel, antiparallel, or parallel and antiparallel D-amino acid strands with interstrand non-covalent bonds; and (b) a target binding region I bound to one terminus of both termini of the structure stabilizing region and a target binding region II bound to the other terminus of the both termini of the structure stabilizing region, wherein the target binding region I and the target binding region II comprises randomly selected n and m D-amino acids, respectively, and wherein the D-Aptide has enhanced stability to a protease with its affinity to the target maintained in comparison with a L-Aptide having the same amino acid sequence with the D-Aptide.

In further another aspect of this invention, there is provided a retro-inverso-Aptamer-Like Peptide (RI-Aptide) having the same D-amino acid sequence with, and the opposite directionality to the D-Aptamer-Like Peptide (D-Aptide), wherein the RI-Aptide has target affinity similar to the D-Aptide.

### BRIEF DESCRIPTION OF THE DRAWINGS

FIG. 1 represents BIAcore X results showing binding kinetic pattern and affinity of D-Aptide1_{EDB} on fibronectin EDB.

FIG. 2 represents circular dichroism (CD) results of L-Aptide1_{EDB} and D-Aptide1_{EDB}.

FIG. 3 represents assay results for stability of L-Aptide1_{EDB} and D-Aptide1_{EDB} to a protease.

FIG. 4 represents a graph showing inhibitory effects of L-Aptide1_{EDB} and D-Aptide1_{EDB} on VEGF activity.

### DETAILED DESCRIPTION

In one aspect of this invention, there is provided a method for enhancing stability of an Aptide consisting of L-amino acids (L-Aptide) to a protease while maintaining the affinity to a target, comprising preparing an Aptamer-Like Peptide (Aptide) comprising: (i) a structure stabilizing region comprising parallel, antiparallel, or parallel and antiparallel amino acid strands with interstrand non-covalent bonds; and (ii) a target binding region I bound to one terminus of both termini of the structure stabilizing region and a target binding region II bound to the other terminus of the both termini of the structure stabilizing region, wherein the target binding region I and the target binding region II comprises randomly selected n and m amino acids, respectively, and wherein the structure stabilizing region, the target binding region I and the target binding region II are prepared with D-amino acids, thereby preparing a D-Aptide or a retro-inverso Aptide.

In another aspect of this invention, there is provided a D-Aptamer-Like Peptide (D-Aptide) which specifically binds to a target, comprising: (a) a structure stabilizing region comprising parallel, antiparallel, or parallel and antiparallel D-amino acid strands with interstrand non-covalent bonds; and (b) a target binding region I bound to one terminus of both termini of the structure stabilizing region and a target binding region II bound to the other terminus of the both termini of the structure stabilizing region, wherein the target binding region I and the target binding region II comprises randomly selected n and m D-amino acids, respectively, and wherein the D-Aptide has enhanced stability to a protease with its affinity to the target maintained in comparison with a L-Aptide having the same amino acid sequence with the D-Aptide.

In further another aspect of this invention, there is provided a retro-inverso-Aptamer-Like Peptide (RI-Aptide) having the same D-amino acid sequence with, and the opposite directionality to the D-Aptamer-Like Peptide (D-Aptide), wherein the RI-Aptide has target affinity similar to the D-Aptide.

The present inventors have made intensive studies to develop technique for improving stability of Aptamer-Like Peptides (Aptides) with its target affinity maintained. As a result, we have discovered that where Aptides were prepared using D-amino acids which were known in the art that they decrease the affinity to a target, prepared D-Aptides have enhanced stability with binding affinity almost identical to L-Aptides (Aptide consisting of L-amino acids).

The term used herein “Aptamer-Like Peptide (Aptide)” refers to BPB peptides disclosed in PCT patent application WO 2010/047515, which were developed by the present inventors. The term ‘BPB’ is changed into “Aptide”. The entire contents of WO 2010/047515 are incorporated by reference herein. The Aptide binds to a specific target like conventional peptide aptamers: However, its structural properties are quite different from the peptide aptamer.

Although WO 2010/047515 describes about the Aptide, there are no any disclosure of Aptides consisting of D-amino acids. Moreover, the main feature of the present invention that Aptides consisting of D-amino acids have enhanced stability with its binding affinity to a target maintained, is not disclosed in WO 2010/047515.

Although the present invention utilizes the structure of the Aptide disclosed in WO 2010/047515, the above-mentioned feature of the present D-Aptide is unusual and inventive, considering a general technical knowledge in the art.

According to a preferred embodiment, a feature of the present invention is related to a discovery that a D-Aptide consisting of D-amino acids and having the same amino acid sequence with L-Aptide consisting of L-amino acids, has largely enhanced stability and binding affinity to a target almost identical to the L-Aptide. According to a general technical knowledge in the art, a target-binding peptide having the D-amino acid sequence identical to a target-binding peptide consisting of L-amino acids is decreased in the affinity to the target (R. C.deL. Milton, et al., Science 256:1445 (1992); S. A. Funke, D. Willbold, Mol. Biosyst. 5:783 (2009)).

On the other hand, in the case of the Aptide structure, D-Aptides having the same amino acid sequence with L-Aptide have almost same target affinity, which is very unusual findings.

The term used herein, “L-Aptide” refers to an Aptide molecule consisting of L-amino acids. The term used herein, “D-Aptide” refers to an Aptide molecule which is consisting of D-amino acids, and has the same amino acid sequence with the L-Aptide.

The expression used to describe the D-Aptide, “maintenance of affinity of L-Aptide to a target” refers that the D-Aptide which is consisting of D-amino acids and has the same amino acid sequence with the L-Aptide, has target affinity substantially identical to that of L-Aptide. The target affinity may be determined as dissociation constant to the target (K_{d}) measured with, for example, SPR (surface plasmon resonance, Smith E A, Corn R M. Surface Plasmon Resonance Imaging as a Tool to Monitor Biomolecular Interactions in an Array Based Format. Appl. Spectroscopy, 2003, 57, 320A-332A). The substantially identical or almost same target affinity between the L-Aptide and D-Aptide indicates that, for example, dissociation constant to a target measured with SPR is substantially same, preferably, a ratio of a dissociation constant (K_{d}) of the D-Aptide to the target to that of the L-Aptide is in a range of 0.1-10, more preferably 0.5-2.0, the most preferably 0.7-1.8.

The term used to describe the D-Aptide, “stability” refers to physical, chemical and biological stability of the D-Aptide, Preferably, biological stability of the D-Aptide. The term, “biological stability” refers to resistance to protease action in vivo after administered in vivo.

The D-Aptide represents enhanced stability against proteases in comparison with the L-Aptide. The stability against proteases may be determined using various technique known in the art. For example, the stability may be determined by time-dependently measuring degradation levels of the D-Aptide after D-Aptides are treated with a representative protease, chymotrypsin or trypsin.

The expression used herein, “D-Aptides have enhanced stability against proteases in comparison with L-Aptide” refers that the degradation level of D-Aptides by the protease is less than or equal to ½, 1/10 or 1/100 in comparison with L-Aptide.

The present invention fundamentally utilizes an Aptide structure disclosed in WO 2010/047515A, and therefore a detailed description of the Aptide will be described hereinbelow.

The structure stabilizing region capable of being utilized in the present invention includes parallel amino acid strands, antiparallel amino acid strands or parallel and antiparallel amino acid strands, and protein structure motifs in which non-covalent bonds are formed by an interstrand hydrogen bond, an electrostatic interaction, a hydrophobic interaction, a Van der Waals interaction, a pi-pi interaction, a cation-pi interaction or a combination thereof. Non-covalent bonds formed by an interstrand hydrogen bond, an electrostatic interaction, a hydrophobic interaction, a Van der Waals interaction, a pi-pi interaction, a cation-pi interaction or a combination thereof contributes to rigidity of a structure stabilizing region.

According to a preferable embodiment, the interstrand non-covalent bonds in the structure stabilizing region include a hydrogen bond, a hydrophobic interaction, a Van der Waals interaction, a pi-pi interaction or a combination thereof.

Alternatively, covalent bond may be involved in the structure stabilizing region. For example, disulfide bond in the structure stabilizing region permits to significantly enhance rigidity of the structure stabilizing region. Increase of rigidity caused by covalent bond is determined according to specificity and affinity of Aptide to a target.

According to a preferable embodiment, amino acid strands of the structure stabilizing region of the present invention are linked by a linker. The term “linker” used herein in the strand refers to a material which may link between strands. For instance, a turn sequence in a β-hairpin used as a structure stabilizing region functions as a linker, and a material (e.g., peptide linker) linking between both C-termini in leucine zipper used as a structure stabilizing region functions as a linker.

Linker may link a parallel amino acid strand, an antiparallel amino acid strand or a parallel and an antiparallel amino acid strands. For example, at least two strands (preferably, two strands) arranged according to a parallel type, at least two strands (preferably, two strands) arranged according to an antiparallel type or at least three strands (preferably, three strands) arranged according to a parallel and an antiparallel type are linked by a linker.

According to a preferable embodiment, the linker of the present invention includes a turn sequence or a peptide linker.

According to a preferable embodiment, the turn sequence of the present invention includes a β-turn, a γ-turn, an α-turn, a π-turn or a ω-loop (Venkatachalam C M (1968), Biopolymers, 6, 1425-1436; Nemethy G and Printz M P. (1972), Macromolecules, 5, 755-758; Lewis P N et al., (1973), Biochim. Biophys. Acta, 303, 211-229; Toniolo C. (1980) CRC Crit. Rev. Biochem., 9, 1-44; Richardson J S. (1981), Adv. Protein Chem., 34, 167-339; Rose G D et al., (1985), Adv. Protein Chem., 37, 1-109; Milner-White E J and Poet R. (1987), TIBS, 12, 189-192; Wilmot C M and Thornton J M. (1988), J. Mol. Biol., 203, 221-232; Milner-White E J. (1990), J. Mol. Biol., 216, 385-397; Pavone V et al. (1996), Biopolymers, 38, 705-721; Rajashankar K R and Ramakumar S. (1996), Protein Sci., 5, 932-946). Most preferably, the turn sequence used in the present invention is a β-turn.

Example of β-turn used as a turn sequence includes preferably type I, type I′, type II, type II′, type III or type III′ turn sequence, more preferably type I, type I′, type II or type II′ turn sequence, much more preferably type I′ or type II′ turn sequence, and most preferably, type I′ turn sequence (B. L. Sibanda et al., J. Mol. Biol., 1989, 206, 4, 759-777; B. L. Sibanda et al., Methods Enzymol., 1991, 202, 59-82).

According to another preferable embodiment, the sequence capable of being used as a turn sequence in the present invention is disclosed in H. Jane Dyson et al., Eur. J. Biochem. 255:462-471 (1998), which is incorporated herein by reference. The sequence capable of being used as a turn sequence in the present invention includes the following amino acid sequence: X-Pro-Gly-Glu-Val; or Ala-X-Gly-Glu-Val (X represents any amino acid selected from 20 amino acids).

According to one embodiment of this invention, it is preferable that two strands arranged according to a parallel type or two strands arranged according to an antiparallel type are linked by a peptide linker in β-sheet or leucine zipper used as a structure stabilizing region in the present invention.

It is possible in the present invention to utilize any peptide linker known to those ordinarily skilled in the art. The sequence of a suitable peptide linker may be selected by considering the following factor: (a) potential to be applied to a flexible extended conformation; (b) inability to form secondary structure capable of interacting with a biological target molecule; (c) absence of a hydrophobic or charged residue which interacts with a biological target molecule. Preferable peptide linkers include Gly, Asn and Ser residue. In addition, other neutral amino acid such as Thr and Ala may be included in a linker sequence. The amino acid sequence suitable in a linker is disclosed in Maratea et al., Gene 40:39-46 (1985); Murphy et al., Proc. Natl. Acad. Sci. USA 83:8258-8562 (1986); U.S. Pat. Nos. 4,935,233, 4,751,180 and 5,990,275. Peptide linker sequence in the present invention may be composed of 1-50 amino acid residues.

According to a preferable embodiment, the structure stabilizing region of the present invention includes a β-hairpin motif, a β-sheet motif linked by a linker or a leucine-zipper motif linked by a linker, more preferably a β-hairpin motif or a β-sheet motif linked by a linker, and most preferably, a β-hairpin motif.

The term “β-hairpin” used herein means the most simple protein motif containing two β strands which are arranged each other in an antiparallel manner. Generally, two β strands in a β-hairpin are linked by a turn sequence.

Preferably, a turn sequence applied to a β-hairpin includes type I, type I′, type II, type II′, type III or type III′ turn sequence, more preferably type I, type I′, type II or type II′ turn sequence, much more preferably type I′ or type II′ turn sequence, and most preferably, type I′ turn sequence. In addition, the following turn sequence may be utilized in a β-hairpin: X-Pro-Gly-Glu-Val; or Ala-X-Gly-Glu-Val (X represents any amino acid selected from 20 amino acids).

According to an illustrative example of the present invention, a type I turn sequence includes Asp-Asp-Ala-Thr-Lys-Thr, and a type I′ turn sequence includes Glu-Asn-Gly-Lys, and a type II turn sequence includes X-Pro-Gly-Glu-Val; or Ala-X-Gly-Glu-Val (X represents any amino acid selected from 20 amino acids), and a type II′ turn sequence includes Glu-Gly-Asn-Lys or Glu-D-Pro-Asn-Lys.

A peptide with β-hairpin conformation is well-known to those ordinarily skilled in the art, for example including tryptophan zipper motif disclosed in U.S. Pat. No. 6,914,123 and Andrea G. Cochran et al., PNAS, 98(10):5578-5583), template-immobilized β-hairpin mimetics in WO 2005/047503 and β-hairpin modifiers in U.S. Pat. No. 5,807,979. Besides, peptide with β-hairpin conformation is disclosed in Smith & Regan (1995) Science 270:980-982; Chou & Fassman (1978) Annu. Rev. Biochem. 47:251-276; Kim & Berg (1993) Nature 362:267-270; Minor & Kim (1994) Nature 367:660-663; Minor & Kim (1993) Nature 371:264-267; Smith et al. Biochemistry (1994) 33:5510-5517; Searle et al. (1995) Nat. Struct. Biol. 2:999-1006; Hague & Gellman (1997) J. Am. Chem. Soc. 119:2303-2304; Blanco et al. (1993) J. Am. Chem. Soc. 115:5887-5888; de Alba et al. (1996) Fold. Des. 1: 133-144; de Alba et al. (1997) Protein Sci. 6:2548-2560; Ramirez-Alvarado et al. (1996) Nat. Struct. Biol. 3:604-612; Stanger & Gellman (1998) J. Am. Chem. Soc. 120:4236-4237; Maynard & Searle (1997) Chem. Commun. 1297-1298; Griffiths-Jones et al. (1998) Chem. Commun. 789-790; Maynard et al. (1998) J. Am. Chem. Soc. 120:1996-2007; and Blanco et al. (1994) Nat. Struct. Biol. 1:584-590, which are incorporated herein by reference.

Most preferably, a peptide with β-hairpin conformation as a structure stabilizing region utilizes a tryptophan zipper motif.

According to a preferable embodiment, the tryptophan zipper used in the present invention is represented by the following Formula I:

X₁-Trp(X₂)X₃-X₄-X₅(X′₂)X₆-X₇ Formula I

wherein X₁ represents Ser or Gly-Glu, and X₂ and X′₂ independently represent Thr, His, Val, Ile, Phe or Tyr, and X₃ represents Trp or Tyr, and X₄ represents type I, type I′, type II, type II′, type III or type III′ turn sequence, and X₅ represents Trp or Phe, and X_{6 represents Trp or Val, and X}₇ represents Lys or Thr-Glu.

More preferably, X₁ represents Ser or Gly-Glu, and X₂ and X′₂ independently represent Thr, His or Val, and X₃ represents Trp or Tyr, and X₄ represents type I, type I′, type II or type II′ turn sequence, and X₅ represents Trp or Phe, and X₆ represents Trp or Val, and X₇ represents Lys or Thr-Glu in the Formula I.

Much more preferably, X₁ represents Ser or Gly-Glu, and X₂ and X′₂ independently represent Thr, His or Val, and X₃ represents Trp, and X₄ represents type I, type I′, type II or type II′ turn sequence, and X₅ represents Trp, and X₆ represents Trp, and X₇ represents Lys or Thr-Glu in the Formula I.

Still much more preferably, X₁ represents Ser, and X₂ and X′₂ represent Thr, and X₃ represents Trp, and X₄ represents type I′ or type II′ turn sequence, and X₅ represents Trp, and X₆ represents Trp, and X₇ represents Lys in the Formula I.

Most preferably, X₁ represents Ser, and X₂ and X′₂ represent Thr, and X₃ represents Trp, and X₄ represents type I′ turn sequence (ENGK) or type II′ turn sequence (EGNK), and X₅ represents Trp, and X₆ represents Trp, and X₇ represents Lys in the Formula I.

An illustrative amino acid sequence of tryptophan zipper suitable in the present invention is described in SEQ ID NOs:1-3 and SEQ ID NOs:5-10.

Another β-hairpin peptide capable of being utilized as a structure stabilizing region in the present invention includes a peptide derived from B1 domain of protein G, i.e. GB1 peptide.

Preferably, the GB1 peptide as a structure stabilizing region used in the present invention is represented by the following Formula II:

X₁-Trp-X₂-Tyr-X₃-Phe-Thr-Val-X₄ Formula II

wherein X₁ represents Arg, Gly-Glu or Lys-Lys, and X₂ represents Gln or Thr, and X₃ represents type I, type I′, type II, type II′, type III or type III′ turn sequence, and X₄ represents Gln, Thr-Glu or Gln-Glu.

More preferably, the structure stabilizing region in the Formula II is represented by the following Formula II′:

X₁-Trp-Thr-Tyr-X₂-Phe-Thr-Val-X₃ Formula II′

wherein X₁ represents Gly-Glu or Lys-Lys, and X₂ represents type I, type I′, type II, type II′, type III or type III′ turn sequence, and X₃ represents Thr-Glu or Gln-Glu.

An exemplified amino acid sequence of GB1 β-hairpin suitable in the present invention is described in SEQ ID NO:4 and SEQ ID NOs:14-15.

Beta-hairpin peptide capable of being utilized as a structure stabilizing region in the present invention includes a HP peptide.

Preferably, the HP peptide as a structure stabilizing region used in the present invention is represented by the following Formula III:

X₁-X₂-X₃-Trp-X₄-X₅-Thr-X₆-X₇ Formula III

wherein X₁ represents Lys or Lys-Lys, and X₂ represents Trp or Tyr, and X₃ represents Val or Thr, and X₄ represents type I, type I′, type II, type II′, type III or type III′ turn sequence, and X₅ represents Trp or Ala, and X₆ represents Trp or Val, and X₂ represents Glu or Gln-Glu.

Still another β-hairpin peptide capable of being utilized as a structure stabilizing region in the present invention is represented by the following Formula IV:

X₁-X₂-X₃-Trp-X₄ Formula IV

wherein X₁ represents Lys-Thr or Gly, and X₂ represents Trp or Tyr, and X₃ represents type I, type I′, type II, type II′, type III or type III′ turn sequence, and X₄ represents Thr-Glu or Gly.

An illustrative amino acid sequence of β-hairpin in Formula III and IV is described in SEQ ID NOs:11-12, SEQ ID NO:15 and SEQ ID NOs:16-19.

According to the present invention, a β-sheet linked by a linker may be used as a structure stabilizing region. The structure of β-sheet includes an extended form of two strands arranged in a parallel or antiparallel manner, preferably in an antiparallel manner, and hydrogen bond is formed between two strands.

Both adjacent termini of two amino acid strands in a β-sheet structure are linked by a linker. As described above, various turn-sequences or peptide linkers may be utilized as a linker. Using a turn sequence as a linker, it is most preferable to utilize a β-turn sequence.

According to another modified embodiment, a leucine zipper motif or a leucine zipper motif linked by a linker may be used as a structure stabilizing region. Leucine zipper motif is a conservative peptide domain which causes a dimerization of two parallel α-chains and a dimerization domain found generally in a protein related to gene expression (“Leucine scissors”. Glossary of Biochemistry and Molecular Biology (Revised). (1997). Ed. David M. Glick. London: Portland Press; Landschulz W H, et al. (1988) Science 240:1759-1764). In general, leucine zipper motif includes a haptad repeat sequence, and a leucine residue is located at fourth or fifth position. For example, a leucine zipper motif capable of being utilized in the present invention includes amino acid sequences such as LEALKEK, LKALEKE, LKKLVGE, LEDKVEE, LENEVAR and LLSKNYH. Practical example of leucine zipper motif used in the present invention is described in SEQ ID NO:39. Half of each leucine zipper motif is composed of a short α-chain, and includes direct leucine interaction between α-chains. In general, leucine zipper motif in a transcription factor consists of a hydrophobic leucine zipper region and basic region (a region interacting with a major groove of DNA molecule). A basic region is not necessary for the leucine zipper motif used in the present invention. In the structure of leucine zipper motif, both adjacent termini of two amino acid strands (i.e., two α-chains) may be linked by a linker. As described above, various turn-sequences or peptide linkers may be utilized as a linker. It is preferable to utilize a peptide linker which has no influence on the structure of leucine zipper motif.

Random amino acid sequence is linked in both termini of the above-mentioned structure stabilizing region. The random amino acid sequence forms a target binding region I and a target binding region II. It is one of the most features of the present invention that a peptide binder is constructed by a bipodal type which a target binding region I and a target binding region II are linked to both termini of a structure stabilizing region, respectively. The target binding region I and the target binding region II bind in a cooperative manner to a target, leading to enhance significantly affinity to the target.

The number (n) of amino acid residues of a target binding region I is not particularly limited, and is an integer of preferably 2-100, more preferably 2-50, much more preferably 2-20 and the most preferably, 3-10.

The number (m) of amino acid residues of a target binding region II is not particularly limited, and is an integer of preferably 2-100, more preferably 2-50, much more preferably 2-20 and the most preferably, 3-10.

The number of amino acid residue of a target binding region I and a target binding region II may be independently different or equivalent. The amino acid sequence of a target binding region I and a target binding region II may be independently different or equivalent, and preferably independently different.

A sequence contained in a target binding region I and/or a target binding region II includes linear or circular amino acid sequence. To enhance stability of peptide sequence in the target binding regions, at least one amino acid residues of amino acid sequence contained in a target binding region I and/or a target binding region II may be modified into an acetyl group, a fluorenyl methoxy carbonyl group, a formyl group, a palmitoyl group, a myristyl group, a stearyl group or a polyethyleneglycol (PEG).

The bipodal-peptide binder of the present invention bound to a biological target molecule may be utilized in: (a) regulation of in vivo physiological response; (b) detection of in vivo material; (c) in vivo molecule imaging; (d) in vitro cell imaging; (e) targeting for drug delivery; and (f) escort molecule.

According to a preferable embodiment, a structure stabilizing region, a target binding region I or a target binding region II (more preferably, a structure stabilizing region and much more preferably, a linker of a structure stabilizing region) further includes a functional molecule. Example of the functional molecule includes a label capable of generating a detectable signal, a chemical drug, a biodrug, a cell penetrating peptide (CPP) and a nanoparticle, but not limited to.

The label capable of generating a detectable signal includes, but is not limited to, T1 contrast materials (e.g., Gd chelate compounds), T2 contrast materials [e.g., superparamagnetic materials (example: magnetite, Fe₃O₄, γ-Fe₂O₃, manganese ferrite, cobalt ferrite and nickel ferrite)], radioactive isotope (example: ¹¹C, ¹⁵O, ¹³N, P³², S³⁵, ⁴⁴Sc, ⁴⁵Ti, ¹¹⁸I, ¹³⁶La, ¹⁹⁸Tl, ²⁰⁰Tl, ²⁰⁵Bi and ²⁰⁶Bi), fluorescent materials (fluorescein, phycoerythrin, rhodamine, lissamine, and Cy3/Cy5), chemiluminescent materials, magnetic particles, mass labels and dense electron particle.

For example, the chemical drug includes an anti-flammatory agent, an analgesic, an anti-arthritic agent, an antispasmodic agent, an anti-depressant, an anti-psychotic agent, a sedative, an anti-anxiety drug, a drug antagonist, an anti-Parkinson's disease drug, a choline agonist, an anti-cancer drug, an anti-angiogenesis inhibitor, an immunosuppressive agent, an anti-viral agent, an antibiotics, an appetite depressant, an anti-choline agent, an anti-histamine agent, an anti-migraine medication, a hormone agent, a coronary, cerebrovascular or perivascular vasodilator, a contraceptive, an anti-thrombotic agent, a diuretic agent, an anti-hypertensive agent, a cardiovascular disease-related therapeutics, a beauty care-related component (e.g., an anti-wrinkle agent, a skin-aging inhibitor and a skin whitening agent), but not limited to.

The above-mentioned biodrug may be insulin, IGF-1 (insulin-like growth factor 1), growth hormone, erythropoietin, G-CSFs (granulocyte-colony stimulating factors), GM-CSFs (granulocyte/macrophage-colony stimulating factors), interferon-α, interferon-β, interferon-γ, interleukin-1α and 1β, interleukin-3, interleukin-4, interleukin-6, interleukin-2, EGFs (epidermal growth factors), calcitonin, ACTH (adrenocorticotropic hormone), TNF (tumor necrosis factor), atobisban, buserelin, cetrorelix, deslorelin, desmopressin, dynorphin A (1-13), elcatonin, eleidosin, eptifibatide, GHRH-II (growth hormone releasing hormone-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclosporin, exedine, lanreotide, LHRH (luteinizing hormone-releasing hormone), nafarelin, parathyroid hormone, pramlintide, T-20 (enfuvirtide), thymalfasin, ziconotide, RNA, DNA, cDNA, antisense oligonucleotide and siRNA, but is not limited to.

The target binding region I and/or target binding region II may include an amino acid sequence capable of binding to various targets. The material to be targeted by the D-Aptide includes a biological target such as a biochemical material, a peptide, a polypeptide, a nucleic acid, a carbohydrate, a lipid, a cell and a tissue, a compound, a metal material or a non-metal material, and preferably, a biological target.

Preferably, the biological target to be bound with the target binding region includes a biochemical material, a peptide, a polypeptide, a glycoprotein, a nucleic acid, a carbohydrate, a lipid or a glycolipid.

For instance, a biochemical material to be bound with the target binding region includes various in vivo metabolites (e.g., ATP, NADH, NADPH, carbohydrate metabolite, lipid metabolite and amino acid metabolite).

An illustrative example of peptide or polypeptide to be bound with the target binding region includes, but is not limited to, an enzyme, a ligand, a receptor, a biomarker, a hormone, a transcription factor, a growth factor, an immunoglobulin, a signal transduction protein, a binding protein, an ionic channel, an antigen, an attachment protein, a structure protein, a regulatory protein, a toxic protein, a cytokine and a coagulation factor. In more detail, a target of a bipodal-peptide binder includes fibronectin extra domain B (ED-B), VEGF (vascular endothelial growth factor), VEGFR (vascular endothelial growth factor receptor), VCAM1 (vascular cell adhesion molecule-1), nAchR (Nicotinic acetylcholine receptor), HAS (Human serum albumin), MyD88, EGFR (Epidermal Growth Factor Receptor), HER2/neu, CD20, CD33, CD52, EpCAM (Epithelial Cell Adhesion Molecule), TNF-α (Tumor Necrosis Factor-α), IgE (Immunoglobulin E), CD11A (α-chain of lymphocyte function-associated antigen 1), CD3, CD25, Glycoprotein IIb/IIIa, integrin, AFP (Alpha-fetoprotein), β2M (Beta2-microglobulin), BTA (Bladder Tumor Antigens), NMP22, cancer antigen 125, cancer antigen 15-3, calcitonin, carcinoembryonic Antigen, chromogranin A, estrogen receptor, progesterone receptor, human chorionic gonadotropin, neuron-specific enolase, PSA (Prostate-Specific Antigen), PAP (Prostatic Acid Phosphatase) and thyroglobulin.

An exemplified example of nucleic acid molecule to be bound with the target binding region includes, but is not limited to, gDNA, mRNA, cDNA, rRNA (ribosomal RNA), rDNA(ribosomal DNA) and tRNA. An illustrative example of carbohydrate to be bound with the target binding region includes cellular carbohydrates such as monosaccharides, disaccharides, trisaccharides and polysaccharides, but is not limited to. An exemplified example of lipid to be bound with the target binding region includes fatty acid, triacylglycerol, sphingolipid, ganglioside and cholesterol, but is not limited to.

The D-Aptide of the present invention may not only be linked to a biomolecule (e.g., protein) exposed on a cell surface but regulate an activity via binding to a biomolecule (e.g., protein) in a cell.

For targeting of cellular protein, it is preferable that the D-Aptide further includes a cell penetrating peptide (CCP).

The above-described CCP includes various CCPs known to those ordinarily skilled in the art, for example HIV-1 tat protein, Tat peptide analogues (e.g., oligoarginine), ANTP peptide, HSV VP22 transcriptional regulatory protein, MTS peptide derived from vFGF, penetratin, transportan or Pep-1 peptide, but is not limited to. The method to bind the CPP to the bipodal-peptide of the present invention may be carried out according to various methods known to those skilled in the art, for example covalently binding CPP to lysine residue of loop region in the structure stabilizing region of the present bipodal-peptide.

There are numerous target proteins which play a critical function in in vivo physiological activity, and the D-Aptide linked to CPP is penetrated into a cell and bound to these target proteins, contributing to regulation (e.g., suppression) of their activities.

As described above, the D-Aptide of the present invention has a “N-target binding region I-one strand of structure stabilizing region-the other strand of structure stabilizing region-target binding region II-C” construct.

According to a preferable embodiment, the D-Aptide of the present invention includes a structure influence inhibiting region which blocks a structural interaction between target binding region and structure stabilizing region and is located at an interspace between target binding region I and one strand of structure stabilizing region and/or between and the other strand of structure stabilizing region and target binding region II. Rotation region of peptide molecule includes an amino acid which φ and ψ rotation are relatively free in peptide molecule. Preferably, an amino acid which φ and ψ rotation are relatively free is glycine, alanine and serine. The number of amino acid in the structure influence inhibiting region of the present invention may be used in a range of 1-10, preferably 1-8 and more preferably 1-3.

The present method comprises the following sub-steps of:

(a) providing a library of the L-Aptide comprising (i) a structure stabilizing region comprising parallel, antiparallel, or parallel and antiparallel L-amino acid strands with interstrand non-covalent bonds; and (ii) a target binding region I bound to one terminus of both termini of the structure stabilizing region and a target binding region II bound to the other terminus of the both termini of the structure stabilizing region, wherein the target binding region I and the target binding region II comprises randomly selected n and m amino acids, respectively;

(b) contacting the library with the target;

(c) selecting a L-Aptide binding to the target;

(d) determining the amino acid sequence of the selected L-Aptide; and

(e) preparing the D-Aptide by substituting L-amino acids of the selected L-Aptide with D-amino acids.

A library of the L-Aptide having the above-described construct may be obtained according to various methods known in the art. The L-Aptide in the library has random sequence. The term “random sequence” used herein means that no sequence preference or no determined (or fixed) amino acid sequence is placed at any position of target binding region I and/or target binding region II.

For example, the library of the L-Aptide may be constructed according to split-synthesis method (Lam et al. (1991) Nature 354:82; WO 92/00091) which is carried out on solid supporter (e.g., polystyrene or polyacrylamide resin).

According to a preferable embodiment, the library of the L-Aptide is constructed by a cell surface display method (e.g., phage display, bacteria display or yeast display). Preferably, the library of the L-Aptide is prepared by a display method based on plasmids, bacteriophages, phagemids, yeasts, bacteria, mRNAs or ribosomes.

Phage display is a technique displaying various polypeptides as proteins fused with coat protein on phage surface (Scott, J. K. and Smith, G. P. (1990) Science 249: 386; Sambrook, J. et al., Molecular Cloning. A Laboratory Manual, 3rd ed. Cold Spring Harbor Press (2001); Clackson and Lowman, Phage Display, Oxford University Press (2004)). Gene of interest is fused with gene III or gene VIII of filamentous phage (e.g., M13), thereby displaying random peptides.

Phagemid may be utilized in phage display. Phagemid is a plasmid vector which has a replication origin of bacteria (e.g., ColE1) and one copy of intergenic region of bacteriophage. DNA fragment cloned into the phagemid is proliferated as same as a plasmid.

Using a phage display method for constructing a library of a L-Aptide, a preferable embodiment of the present invention includes the steps of: (i) preparing a library of an expression vector including a fusion gene in which a gene encoding a phage coat protein (e.g., gene III or gene VIII coat protein of filamentous phage such as M13) is fused with a gene encoding a bipodal-peptide binder, and a transcriptional regulatory sequence (e.g., lac promoter) operatively linked to the fusion gene; (ii) introducing the library into a suitable host cell; (iii) displaying a fusion protein on the phage surface by culturing the host cell and forming a recombinant phage or a phagemid virus particle; (iv) binding the particle to a target molecule by contacting the virus particle with a biological target molecule; and (v) removing the particle unbound to the target molecule.

The method to construct and screen a peptide library using a phage display method is disclosed in U.S. Pat. Nos. 5,723,286, 5,432,018, 5,580,717, 5,427,908, 5,498,530, 5,770,434, 5,734,018, 5,698,426, 5,763,192 and 5,723,323.

The method to prepare an expression vector including a Aptamer-like peptide gene may be carried out according to the method known in the art. For example, expression vector may be prepared by inserting a L-Aptide into a public phagemid or phage vector (e.g., pIGT2, fUSES, fAFF1, fd-CAT1, m663, fdtetDOG, pHEN1, pComb3, pComb8, pCANTAB 5E (Pharmacia), LamdaSurfZap, pIF4, PM48, PM52, PM54, fdH and p8V5).

Most phage display methods are carried out using filamentous phage. Additionally, a library of L-Aptide may be constructed using lambda phage display (WO 95/34683; U.S. Pat. No. 5,627,024), T4 phage display (Ren et al. (1998) Gene 215:439; Zhu (1997) CAN 33:534) and T7 phage display (U.S. Pat. No. 5,766,905).

The method to introduce a vector library into a suitable host cell may be performed according to various transformation methods, and most preferably, electroporation (See, U.S. Pat. Nos. 5,186,800, 5,422,272 and 5,750,373). The host cell suitable in the present invention includes gram-negative bacteria such as E. coli which includes JM101, E. coli K12 strain 294, E. coli strain W3110 and E. coli XL-1Blue (Stratagene), but is not limited to. It is preferable that host cells are prepared as a competent cell before transformation (Sambrook, J. et al., Molecular Cloning. A Laboratory Manual, 3rd ed. Cold Spring Harbor Press (2001)). In general, selection of transformed cells may be carried out by culturing cells in a medium containing antibiotics (e.g., tetracycline and ampicillin). Selected transformants are further cultured in the presence of helper phage to produce recombinant phages or phagemid virus particles. Suitable helper phage as described above includes, but is not limited to, Ex helper phage, M13-KO7, M13-VCS and R408.

Selection of virus particle binding to a biological target molecule may be carried out using a conventional biopanning process (Sambrook, J. et al., Molecular Cloning. A Laboratory Manual, 3rd ed. Cold Spring Harbor Press (2001); Clackson and Lowman, Phage Display, Oxford University Press (2004)).

Amino acid sequences of the target-affinity L-APtides selected from the above process are determined. The sequence determination of L-Aptide may be performed by methods known in the art (Hanno Steen & Matthias Mann. The abc's (and xyz's) of peptide sequencing. Nature Reviews Molecular Cell Biology, 5:699-711, 2004).

Finally, D-Aptides are prepared by substituting L-amino acids of L-Aptide with D-amino acids. The preparation of D-Aptides may be performed using chemical synthesis techniques, especially, solid-phase synthesis techniques (Merrifield, J. Amer Chem. Soc. 85:2149-54 (1963); Stewart, et al., Solid Phase Peptide Synthesis, 2nd. ed., Pierce Chem. Co.: Rockford, 111 (1984)).

Practical examples of the D-Aptide of the present invention are described in SEQ ID NOs:20-38 and SEQ ID NOs:40-42, where the amino acids described in the sequence listing are D-amino acids.

The D-Aptide prepared by the present invention has target affinity substantially identical to L-Aptide. Preferably, a ratio of a dissociation constant (K_{d}) of the D-Aptide to a target to that of the L-Aptide is in a range of 0.7-2.5, more preferably 0.8-2.0, the most preferably 0.85-1.8.

According to a preferred embodiment, a dissociation constant (K_{d}) of the D-Aptide to a target is in a range of 0.01-500 nM, more preferably 0.1-300 nM, much more preferably 1-200 nM, much more preferably 10-200 nM, and hence the D-Aptide shows very high affinity to the target.

According to a preferred embodiment, the D-Aptide has the amino acid sequence and directionality identical to those of the L-Aptide.

Alternatively, the D-Aptide has the same amino acid sequence with, and opposite directionality to the L-Aptide. Such peptides are a retro inverso (RI)-Aptide. Where RI-Aptides are prepared by referring to the amino acid sequence of the L-Aptide, the prepared RI-Aptide has also enhanced stability to a protease and target affinity substantially identical to L-Aptide, like D-Aptide.

The D-Aptide of the present invention exhibits the K_{D} value (dissociation constant) of a very low level (for example, nM level) and, therefore, exhibits very high affinity toward a biological target molecule.

The D-Aptide of the present invention has applications not only in pharmaceuticals and detection of in vivo material but also in in vivo imaging, in vitro cell imaging, and drug delivery targeting, and can be very usefully employed as an escort molecule.

Especially, the D-Aptide of the present invention has largely enhanced stability against proteases with its target affinity maintained in comparison with L-Aptides, and therefore improves a chance of applying Aptide molecules as pharmaceuticals.

The features and advantages of the present invention will be summarized as follows:

(a) The present invention provides a D-Aptide having largely enhanced stability with its target affinity maintained.

(b) Unlike a general technical knowledge in the art, the D-Aptide of the present invention has largely enhanced stability and substantially same target affinity with L-Aptide.

(c) The D-Aptide of the present invention exhibits the K_{D} value (dissociation constant) of a very low level (for example, nM level) and, therefore, exhibits very high affinity toward a biological target molecule.

(d) The D-Aptide of the present invention has applications not only in pharmaceuticals and detection of in vivo material but also in in vivo imaging, in vitro cell imaging, and drug delivery targeting, and can be very usefully employed as an escort molecule.

(e) Especially, the D-Aptide of the present invention has largely enhanced stability against proteases with its target affinity maintained in comparison with L-Aptides, and therefore improves a chance of applying Aptide molecules as pharmaceuticals.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Experimental Method

### Preparation of L-Form, D-Form and Retro Inverso-Form of Aptide1_{EDB} and Measurement of their Affinity

To investigate whether the activity is maintained where Aptamer-Like Peptides specifically binding to fibronectin EDB, i.e., L-Aptide1_{EDB} are converted into D- and Retro inverso (RI)-form, their affinity to a target was measured. Firstly, 3 peptides, L-Aptide1_{EDB} (HCSSAVGSWTWENGKWTWKGIIRLEQ, all L-amino acids, SEQ ID NO:21), D-Aptide1_{EDB} (HCSSAVGSWTWENGKWTWKGIIRLEQ, all D-amino acids, SEQ ID NO:21) and RI-Aptide1_{EDB} (QELRIIGKWTWKGNEWTWSGVASSCH, all D-amino acids), were synthesized by Anigen Inc. (Korea). Their affinity for a target was determined using BIAcore X (Biacore AB, Uppsala, Sweden). Biotin-EDB of 2000 RU was immobilized on streptavidin SA chip (Biacore). A PBS (pH 7.4) was used as a running buffer, a flow rate was 30 μl/min, and the affinity was determined with BIAevaluation software (Biacore AB, Uppsala, Sweden) after measuring kinetics of various concentrations.

### Preparation of L-Form, D-Form and Retro Inverso-Form of Aptide1_{VEGF} and Measurement of their Affinity

To investigate whether the activity is maintained where Aptamer-Like Peptides specifically binding to VEGF (Vascular endothelial growth factor), i.e., L-Aptide1_{VEGF} are converted into D- and Retro inverso (RI)-form, their affinity to a target was measured. Firstly, 3 peptides, L-Aptide1_{VEGF} (HANFFQGSWTWENGKWTWKGWKYNQS, all L-amino acids, SEQ ID NO:23), D-Aptide1_{VEGF} (HANFFQGSWTWENGKWTWKGWKYNQS, all D-amino acids, SEQ ID NO:23) and RI-Aptide1_{VEGF} (SQNYKWGKWTWKGNEWTWSGQFFNAH, all D-amino acids), were synthesized by Anigen Inc. (Korea). Their affinity for a target was determined using BIAcore X (Biacore AB, Uppsala, Sweden). VEGF₁₂₁ of 3000 RU was immobilized on CM5 chip (Biacore). A PBS (pH 7.4) was used as a running buffer, a flow rate was 30 μl/min, and the affinity was determined with BIAevaluation software (Biacore AB, Uppsala, Sweden) after measuring kinetics of various concentrations.

### Preparation of L-Form, D-Form and Retro Inverso Form of Aptide_{HSA} and Measurement of their Affinity

To investigate whether the activity is maintained where Aptamer-Like Peptides specifically binding to HSA (Human serum albumin), i.e., L-Aptide_{HSA} are converted into D- and Retro inverso (RI)-form, their affinity to a target was measured. Firstly, 3 peptides, L-Aptide_{HSA} (HAHFNFGSWTWENGKWTWKGIWLPAR, all L-amino acids, SEQ ID NO:42), D-Aptide_{HSA} (HAHFNFGSWTWENGKWTWKGIWLPAR, all D-amino acids, SEQ ID NO:42) and RI-Aptide_{HSA} (RAPLWIGKWTWKGNEWTWSGFNFHAH, all D-amino acids), were synthesized by Anigen Inc. (Korea). Their affinity for a target was determined using BIAcore X (Biacore AB, Uppsala, Sweden). HSA of 3000 RU was immobilized on CM5 chip (Biacore). A PBS (pH 7.4) was used as a running buffer, a flow rate was 30 μl/min, and the affinity was determined with BIAevaluation software (Biacore AB, Uppsala, Sweden) after measuring kinetics of various concentrations.

Circular Dichroism (CD)

To investigate CD patterns of L- or D-Aptide1_{EDB}, spectra of the L- or D-Aptide1_{EDB} of 300 μl/ml in 20 mM potassium phosphate (pH7) were obtained using 0.1 mm cells. Jasco J-810 spectopolarimeter was used as a CD device, and the spectra were obtained from 190-250 nm.

Analysis for the Degradation of Aptimer-Like Peptides by Proteases

Degradation levels of the L-Aptide1_{EDB} and D-Aptide1_{EDB} were measured using proteases, α-chymotrypsin and trypsin (Sigma). The proteases of 10 unit were added to 100 μg peptides, followed by the reaction for 30, 60, 120 and 720 min, and the reaction was blocked by adding SDS sample buffer (Invitrogen). Subsequently, results for the peptide degradation were obtained from SDS-PAGE.

Analysis for Inhibitory Activity of L- or D-Aptide1_{VEGF} Against VEGF

HUVECs (Human Umbilical Vein Endothelial Cells, ATCC) were starved in M199 medium (Gibco) supplemented with 1% FBS, and then they were attached to a 96-well plate at 5000 cell/well. After 12 hours, the cells were treated with medium containing both various concentrations of L-Aptide1_{VEGF} and D-Aptide1_{VEGF}, and VEGF₁₆₅ (R&D Systems). After 72 hours, the growth inhibition rate of HUVECs by the L-Aptide_{VEGF} and D-Aptide_{VEGF} was determined using EZ-cytox cell viability assay kit (Daeil Labservice, Korea).

### Experimental Results

Affinity of L- or D-Aptide1_{EDB} for Fibronectin EDB

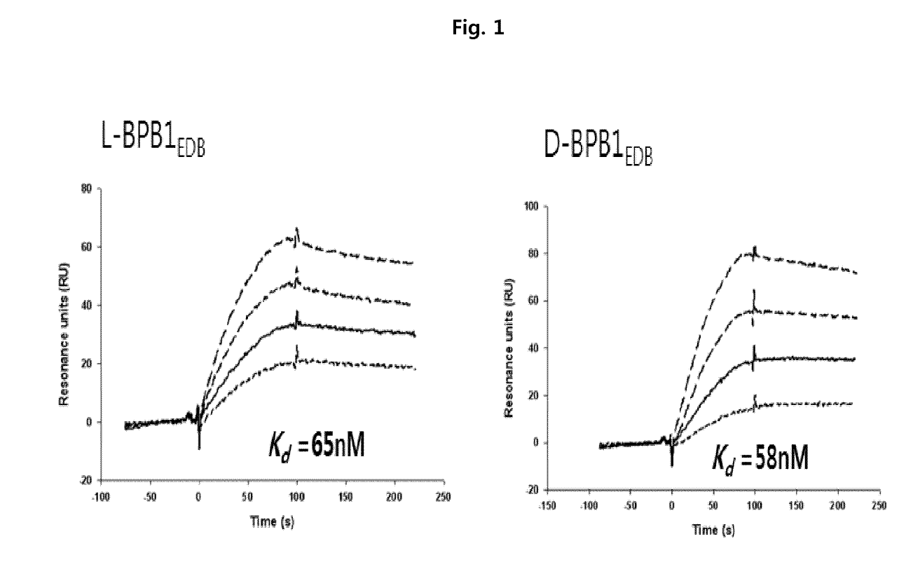

Affinity of the L-Aptide1_{EDB} and D-Aptide1_{EDB} for Fibronectin EDB was determined with BIAcore X. As shown in FIG. 1, the L-Aptide1_{EDB} and D-Aptide1_{EDB} showed almost same kinetics properties, and their dissociation constant was also almost same (each 65 nM and 58 nM), indicating that their affinity for Fibronectin EDB was almost identical. Given that it is well-known in the art that the affinity of peptides consisting of D-form amino acids to a target is generally decreased, these results are very surprising.

The Affinity of L-, D- and RI-Aptide1_{EDB}; D- and RI-Aptide1_{VEGF}; and L-, D- and RI-Aptide_{HSA}

The affinity of L-, D- and RI-form of Aptide1_{EDB} specifically binding to fibronectin EDB, Aptide1_{VEGF} specifically binding to VEGF, and Aptide_{HSA} specifically binding to HAS was measured by SPR (surface plasmon resonance). As shown in Table 1, the D-Aptides were bound to their target with affinity similar to L-Aptide. In the case of RI-form, RI-Aptide1_{EDB} and RI-Aptide_{HSA} were bound to their target with affinity similar to L-Aptide, whereas RI-Aptide1_{VEGF} were barely bound to VEGF as a target.

TABLE 1

| Target | Aptide | kₐ [M⁻¹s⁻¹] | k_{d} [s⁻¹] | K_{d} [M] |
|---|---|---|---|---|
| EDB | L-Aptide1_{EDB} | 1.0 × 10⁴ | 9.5 × 10⁻⁴ | 65 × 10⁻⁹ |
| | D-Aptide1_{EDB} | 1.5 × 10⁴ | 9.0 × 10⁻⁴ | 58 × 10⁻⁹ |
| | RI-Aptide1_{EDB} | 1.9 × 10⁴ | 1.1 × 10⁻³ | 58 × 10⁻⁹ |
| VEGF | L-Aptide1_{VEGF} | 3.5 × 10⁵ | 1.0 × 10⁻² | 30 × 10⁻⁹ |
| | D-Aptide1_{VEGF} | 1.3 × 10⁵ | 6.6 × 10⁻³ | 50 × 10⁻⁹ |
| | RI-Aptide1_{VEGF} | Non-binding | Non-binding | Non-binding |
| HSA | L-Aptide_{HSA} | 9.5 × 10⁴ | 1.3 × 10⁻² | 142 × 10⁻⁹ |
| | D-Aptide_{HSA} | 9.7 × 10⁴ | 1.4 × 10⁻² | 153 × 10⁻⁹ |
| | RI-Aptidens_{HSA} | 5.8 × 10⁴ | 8.2 × 10⁻³ | 141 × 10⁻⁹ |

Kinetic binding data of the L-, D- and RI-Aptide against EDB, VEGF and HAS

Circular Dichroism

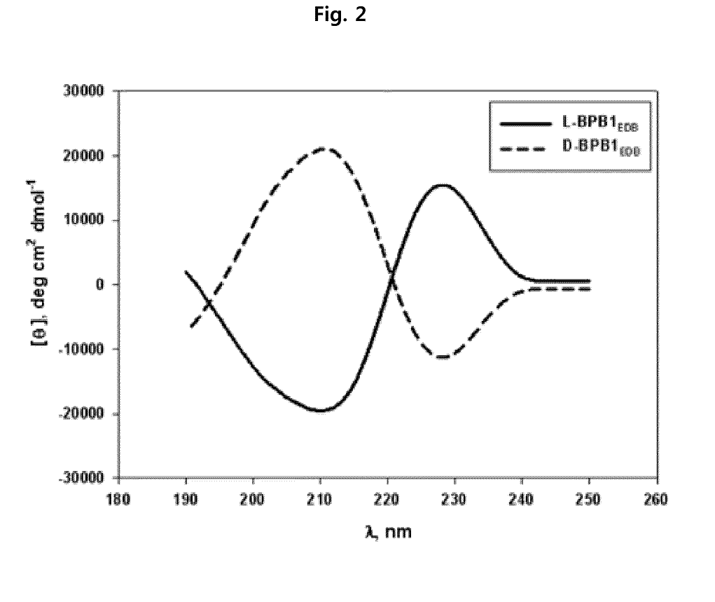

The circular dichroism (CD) for L-Aptide1_{EDB} and D-Aptide1_{EDB} was conducted at 190-250 nm. As shown in FIG. 2, the CD spectrum pattern of L-Aptide1_{EDB} was similar to that of trpzip which was a structural scaffold of L-Aptide1_{EDB}. As expected, D-Aptide1_{EDB} showed a reciprocal spectrum of L-Aptide1_{EDB}.

Protease Assay

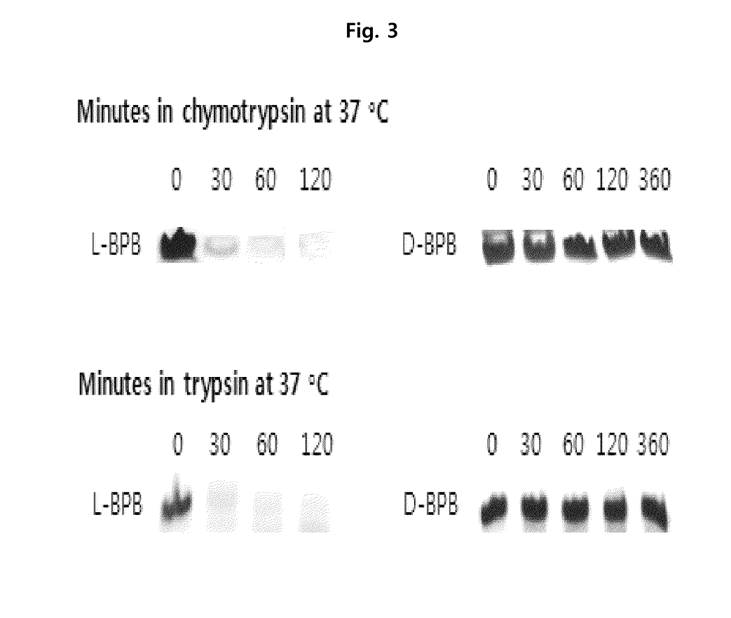

A degree of degradation of L-Aptide1_{EDB} and D-Aptide1_{EDB} was measured using proteases, α-chymotrypsin and trypsin. As shown in FIG. 3, all L-Aptide1_{EDB} was degraded within 30 min by the treatment of α-chymotrypsin or trypsin, whereas the D-Aptide1_{EDB} was not degraded until even 360 min. These results indicate that D-Aptide1_{EDB} has a good tolerance to the degradation by proteases, and excellent in vivo stability when administered in vivo.

Assay for Inhibition of VEGF Activity (HUVEC Proliferation Assay)

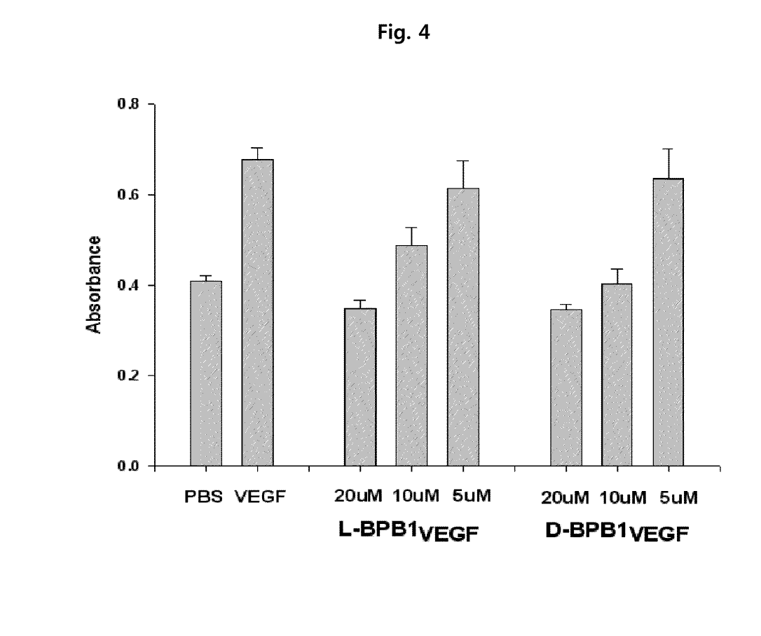

HUVEC proliferation assay was performed to examine whether L-Aptide1_{VEGF} and D-Aptide1_{VEGF} inhibit VEGF activity. VEGF is a growth hormone to promote growth of epithelial cells, and thus the growth of epithelial cells can be inhibited via the inhibition of VEGF. As shown in FIG. 4, where 20 μM, 10 μM and 5 μM L-Aptide1_{VEGF}, and 20 μM, 10 μM and 5 μM D-Aptide1_{VEGF} were treated, they completely inhibited VEGF activity at 20 μM. Moreover, at 10 μM, D-form more inhibited VEGF activity than L-form. It is considered that these results come from stability of D-form against proteases higher than L-form.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A method for enhancing stability of an Aptide consisting of L-amino acids (L-Aptide) to a protease while maintaining the affinity to a target, comprising preparing an Aptamer-Like Peptide (Aptide) comprising: (i) a structure stabilizing region comprising parallel, antiparallel, or parallel and antiparallel amino acid strands with interstrand non-covalent bonds; and (ii) a target binding region I bound to one terminus of both termini of the structure stabilizing region and a target binding region II bound to the other terminus of the both termini of the structure stabilizing region, wherein the target binding region I and the target binding region II comprises randomly selected n and m amino acids, respectively,
wherein the structure stabilizing region, the target binding region I and the target binding region II are prepared with D-amino acids, thereby preparing a D-Aptide or a retro-inverso Aptide.

2. The method of claim 1, wherein a ratio of a dissociation constant (K_{d}) of the D-Aptide or retro-inverso Aptide to the target to that of the L-Aptide is in a range of 0.1-10.

3. The method of claim 2, wherein the ratio of a dissociation constant (K_{d}) of the D-Aptide or retro-inverso Aptide to the target to that of the L-Aptide is in a range of 0.5-2.0.

4. The method of claim 1, wherein the interstrand non-covalent bonds is a hydrogen bond, an electrostatic interaction, a hydrophobic interaction, a Van der Waals interaction, a pi-pi interaction, a cation-pi interaction or a combination thereof.

5. The method of claim 1, wherein the amino acid strands in the structure stabilizing region are linked by a linker.

6. The method of claim 1, wherein the structure stabilizing region is a β-hairpin, a hairpin, a β-sheet linked by a linker, a leucine-zipper, a leucine-zipper linked by a linker, a leucine-rich motif or a leucine-rich motif linked by a linker.

7. The method of claim 1, wherein the target binding region I and target binding region II bind in a cooperative manner to the target.

8. The method of claim 1, wherein the structure stabilizing region, the target binding region I or the target binding region II further comprises a functional molecule.

9. The method of claim 1, which comprises sub-steps of:
(a) providing a library of the L-Aptide comprising (i) a structure stabilizing region comprising parallel, antiparallel, or parallel and antiparallel L-amino acid strands with interstrand non-covalent bonds; and (ii) a target binding region I bound to one terminus of both termini of the structure stabilizing region and a target binding region II bound to the other terminus of the both termini of the structure stabilizing region, wherein the target binding region I and the target binding region II comprises randomly selected n and m amino acids, respectively;
(b) contacting the library with the target;
(c) selecting a L-Aptide binding to the target;
(d) determining the amino acid sequence of the selected L-Aptide; and
(e) preparing the D-Aptide or the retro-inverso Aptide by substituting L-amino acids of the selected L-Aptide with D-amino acids.

10. The method of claim 9, the D-Aptide has the amino acid sequence and directionality identical to those of the L-Aptide.

11. The method of claim 9, the retro-inverso Aptide has opposite directionality to the L-Aptide.

12. A D-Aptide or retro-inverso Aptide which specifically binds to a target, comprising: (a) a structure stabilizing region comprising parallel, antiparallel, or parallel and antiparallel D-amino acid strands with interstrand non-covalent bonds; and (b) a target binding region I bound to one terminus of both termini of the structure stabilizing region and a target binding region II bound to the other terminus of the both termini of the structure stabilizing region, wherein the target binding region I and the target binding region II comprises randomly selected n and m D-amino acids, respectively.

13. The D-Aptide or retro-inverso Aptide of claim 12, wherein the D-Aptide or retro-inverso Aptide has enhanced stability to a protease with its affinity to the target maintained in comparison with a L-Aptide having the amino acid sequence of the same or opposite direction to the D-Aptide or retro-inverso Aptide.

14. The D-Aptide or retro-inverso Aptide of claim 12, wherein a ratio of a dissociation constant (K_{d}) of the D-Aptide or the retro-inverso Aptide to the target to that of the L-Aptide is in a range of 0.1-10.

15. The D-Aptide or retro-inverso Aptide of claim 14, wherein the ratio of a dissociation constant (K_{d}) of the D-Aptide or the retro-inverso Aptide to the target to that of the L-Aptide is in a range of 0.5-2.0.

16. The D-Aptide or retro-inverso Aptide of claim 12, wherein the interstrand non-covalent bonds is a hydrogen bond, an electrostatic interaction, a hydrophobic interaction, a Van der Waals interaction, a pi-pi interaction, a cation-pi interaction or a combination thereof.

17. The D-Aptide or retro-inverso Aptide of claim 12, wherein the amino acid strands in the structure stabilizing region are linked by a linker.

18. The D-Aptide or retro-inverso Aptide of claim 12, wherein the structure stabilizing region is a β-hairpin, a hairpin, a β-sheet linked by a linker, a leucine-zipper, a leucine-zipper linked by a linker, a leucine-rich motif or a leucine-rich motif linked by a linker.

19. The D-Aptide or retro-inverso Aptide of claim 12, wherein the target binding region I and target binding region II bind in a cooperative manner to the target.

20. The D-Aptide or retro-inverso Aptide of claim 12, wherein the structure stabilizing region, the target binding region I or the target binding region II further comprises a functional molecule.


SEQUENCE LISTING (42 sequences)

SEQ ID NO: 1 - protein, 12 aa, Artificial Sequence
  Trpzip1
SWTWEGNKWTWK

SEQ ID NO: 2 - protein, 12 aa, Artificial Sequence
  Trpzip2
SWTWENGKWTWK

SEQ ID NO: 3 - protein, 12 aa, Artificial Sequence
  Trpzip3
SWTWEPNKWTWK

SEQ ID NO: 4 - protein, 16 aa, Artificial Sequence
  gb1, 41-56
GEWTYDDATKTFTVTE

SEQ ID NO: 5 - protein, 16 aa, Artificial Sequence
  Trpzip4
GEWTWDDATKTWTWTE

SEQ ID NO: 6 - protein, 16 aa, Artificial Sequence
  Trpzip5
GEWTYDDATKTFTWTE

SEQ ID NO: 7 - protein, 16 aa, Artificial Sequence
  Trpzip6
GEWTWDDATKTWTVTE

SEQ ID NO: 8 - protein, 16 aa, Artificial Sequence
  Trpzip7
GEWHWDDATKTWVWTE

SEQ ID NO: 9 - protein, 16 aa, Artificial Sequence
  Trpzip8
GEWVWDDATKTWHWTE

SEQ ID NO: 10 - protein, 16 aa, Artificial Sequence
  Trpzip9
GEWVWDDATKTWVWTE

SEQ ID NO: 11 - protein, 12 aa, Artificial Sequence
  (T3V)-HP6
KYVWSNGKWTVE

SEQ ID NO: 12 - protein, 12 aa, Artificial Sequence
  Espinosa-GB1(b)
RWQYVNGKFTVQ

SEQ ID NO: 13 - protein, 16 aa, Artificial Sequence
  GB1m2
GEWTYNPATGKFTVTE

SEQ ID NO: 14 - protein, 16 aa, Artificial Sequence
  GB1m3
KKWTYNPATGKFTVQE

SEQ ID NO: 15 - protein, 16 aa, Artificial Sequence
  HP5W4
KKWTYNPATGKWTWQE

SEQ ID NO: 16 - protein, 16 aa, Artificial Sequence
  HP5W
KKYTWNPATGKWTVQE

SEQ ID NO: 17 - protein, 16 aa, Artificial Sequence
  HP5A
KKYTWNPATGKATVQE

SEQ ID NO: 18 - protein, 12 aa, Artificial Sequence
  HP7
KTWNPATGKWTE

SEQ ID NO: 19 - protein, 10 aa, Artificial Sequence
  Chignolin
GYDPETGTWG

SEQ ID NO: 20 - protein, 26 aa, Artificial Sequence
  BPB-EB-B1
MSADKSGSWTWENGKWTWKGQVRTRD

SEQ ID NO: 21 - protein, 26 aa, Artificial Sequence
  BPB-ED-B2
HCSSAVGSWTWENGKWTWKGIIRLEQ

SEQ ID NO: 22 - protein, 26 aa, Artificial Sequence
  BPB-ED-B3
HSQGSPGSWTWENGKWTWKGRYSHRA

SEQ ID NO: 23 - protein, 26 aa, Artificial Sequence
  BPB-VEGF1
HANFFQGSWTWENGKWTWKGWKYNQS

SEQ ID NO: 24 - protein, 26 aa, Artificial Sequence
  BPB-VEGF2
ASPFWAGSWTWENGKWTWKGWVPSNA

SEQ ID NO: 25 - protein, 26 aa, Artificial Sequence
  BPB-VEGF3
HAFYYTGSWTWENGKWTWKGWPVTTS

SEQ ID NO: 26 - protein, 26 aa, Artificial Sequence
  BPB-VEGF4
YGAYPWGSWTWENGKWTWKGWRVSRD

SEQ ID NO: 27 - protein, 26 aa, Artificial Sequence
  BPB-VEGF5
AAPTSFGSWTWENGKWTWKGWQMWHR

SEQ ID NO: 28 - protein, 26 aa, Artificial Sequence
  BPB-VCAM1
QARDCTGSWTWENGKWTWKGPSICPI

SEQ ID NO: 29 - protein, 26 aa, Artificial Sequence
  BPB-nAchR1
EASFWLGSWTWENGKWTWKGKGTLNR

SEQ ID NO: 30 - protein, 26 aa, Artificial Sequence
  BPB-nAchR2
YAYPLLGSWTWENGKWTWKGWYQKWI

SEQ ID NO: 31 - protein, 26 aa, Artificial Sequence
  BPB-nAchR3
ASLPAWGSWTWENGKWTWKGWSTRTA

SEQ ID NO: 32 - protein, 26 aa, Artificial Sequence
  BPB-BSA1
AASPYKGSWTWENGKWTWKGGWRMKM

SEQ ID NO: 33 - protein, 26 aa, Artificial Sequence
  BPB-BSA2
SANSLYGSWTWENGKWTWKGTSRQRW

SEQ ID NO: 34 - protein, 26 aa, Artificial Sequence
  BPB-BSA3
YAHVYYGSWTWENGKWTWKGHRVTQT

SEQ ID NO: 35 - protein, 26 aa, Artificial Sequence
  BPB-BSA4
YGAYPWGSWTWENGKWTWKGWRVSRD

SEQ ID NO: 36 - protein, 26 aa, Artificial Sequence
  BPB-BSA5
YAHFGWGSWTWENGKWTWKGTTDSQS

SEQ ID NO: 37 - protein, 26 aa, Artificial Sequence
  BPB-MyD88-1
HSHAFYGSWTWENGKWTWKGNPGWWT

SEQ ID NO: 38 - protein, 26 aa, Artificial Sequence
  BPB-MyD88-2
ASTINFGSWTWENGKWTWKGYTRRWN

SEQ ID NO: 39 - protein, 35 aa, Artificial Sequence
  Leucine zipper for BPB
GGSMKQLEDKVEELLSKNYHLENEVARLKKLVGER

SEQ ID NO: 40 - protein, 41 aa, Artificial Sequence
  BPBLeu-ED-B-1
CSSPIQGGSMKQLEDKVEELLSKNYHLENEVARLKKLVGER

SEQ ID NO: 41 - protein, 41 aa, Artificial Sequence
  BPBLeu-ED-B-2
IIRLEQGGSMKQLEDKVEELLSKNYHLENEVARLKKLVGER

SEQ ID NO: 42 - protein, 26 aa, Artificial Sequence
  BPB-HSA
HAHFNFGSWTWENGKWTWKGIWLPAR